(19) United States
(12) Patent Application Publication
(10) Pub. No.: US 2022/0378348 A1
(43) Pub. Date: Dec. 1, 2022

(54) PORTABLE UROFLOWMETRY APPARATUS, UROFLOWMETRY AND CREATING MICTURITION CHART SYSTEM USING THE SAME, AND UROFLOWMETRY METHOD

(71) Applicant: SapienMed Co., Ltd, Seoul (KR)

(72) Inventors: Seung June OH, Seoul (KR); Ji Hoon PARK, Seoul (KR)

(73) Assignee: SapienMed Co., Ltd, Seoul (KR)

(21) Appl. No.: 17/571,509

(22) Filed: Jan. 9, 2022

Related U.S. Application Data
(60) Provisional application No. 63/193,607, filed on May 27, 2021.

(51) Int. Cl. A61B 5/20; A61B 5/00; G16H 50/20

(52) U.S. Cl. CPC A61B 5/208; A61B 5/742; A61B 5/0004; G16H 50/20; A61B 2560/0431

(57) ABSTRACT

The present disclosure relates to a portable uroflowmetry apparatus, and uroflowmetry, voiding diary writing system and uroflowmetry method using the same.

### FIELD

The present disclosure relates to a portable uroflowmetry apparatus, uroflowmetry and creating micturition chart system using the same, and uroflowmetry method, and more particularly, to a portable uroflowmetry apparatus, that allows to easily write voiding information using the portable uroflowmetry apparatus, and to automatically write voiding diary using the portable uroflowmetry apparatus, and uroflowmetry and voiding diary writing system using the same, and uroflowmetry method.

### BACKGROUND

A urinary tract refers to the entire passage through which urine is produced and discharged out of the body, and generally includes a kidney, ureter, urinary bladder and urethra. Further, a lower urinary tract collectively refers to the biostructures and organs from the bladder down, which urine passes through, including the urethra including the urethral sphincter and urethral meatus. Urination is a basic physiological activity that people perform several times a day, and the function of the lower urinary tract undergoes gradual changes throughout life according to changes in end organs such as the bladder and urethral sphincter and the nervous system that controls this function.

Dysfunction of such a lower urinary tract is a symptom that can be observed from an examinee due to various diseases such as benign prostatic hyperplasia, neurogenic bladder, overactive bladder, urinary incontinence, nocturia, urethral stricture, and acquired voiding dysfunction. Diagnosing a lower urinary tract dysfunction involves selectively performing history taking, physical examination, voiding diary, postvoid residual volume measurement, imaging study, cystourethroscopy, and urodynamic study. The purpose of all these examinations is basically to accurately estimate the lower urinary tract dysfunction and symptoms during the patient's everyday life, and thus it is most important to accurately reproduce and record the usual voiding pattern.

Urodynamic study is a collective term for examinations made up of several detailed items such as uroflowmetry, filling cystometry, pressure-flow study, urethral pressure profile, and electromyography of the urethral sphincter. These examinations are conducted selectively depending on the examinee having symptoms of the lower urinary tract dysfunction. Uroflowmetry is an examination where the patient urinates on a measurement apparatus, and diagnostic parameters related to the urinary flow rate are calculated, and the voiding curve pattern over time is visually shown. Since uroflowmetry is non-invasive, it is widely utilized as a screening test to be primarily conducted on patients with urinary tract dysfunctions. Therefore, although uroflowmetry can also be conducted as part of a urodynamic study, it is far more commonly utilized independently by a stand-alone uroflowmetry measurement apparatus.

However, there are several practical problems that accompany a general uroflowmetry. First, according to prior art, the examinee has to visit a hospital, and must be examined in front of a uroflowmetry equipment installed in a urodynamic suite provided in the hospital. Usually, the examination itself is considered valid only when the patient's voided volume is at least a certain amount (120-150 ml). However, if the voided volume is less than that, it becomes difficult to interpret the examination results, and thus a repeat test is required. Therefore, cases occur where it is difficult to make a diagnostic determination promptly. Second, there is a problem of limitation of examination venue. That is, examinees are instructed to void in front of an artificial uroflowmetry apparatus, but considering the fact that voiding is a highly private physiological activity that is greatly affected psychologically, there is a disadvantage that it is difficult to implement voiding that accurately reflects the examinee's usual pattern in such an artificial environment. Third, there is a disadvantage that there are many variations in voiding patterns. Therefore, it is general to conduct uroflowmetry at least two times, and interpret the best result of the two as the representative value.

Accordingly, uroflowmetry requires conditions that would not affect the examinee's psychological state so much and where the usual voiding pattern can be well revealed. Therefore, there is a demand for the development of uroflowmetry system that can obtain examination results anytime and anywhere, not only in a specific place in a hospital, but also at home or in a public toilet, while the patient is living his or her natural daily life.

Voiding flow rate and voiding curve pattern may show some variations at every urination even in one individual. However, there are some common patterns that are distinct for each individual and each disease. Therefore, it is difficult to consider that a result is representative of the usual urination pattern with only one examination for each individual. In order to accurately reproduce a typical and universal voiding pattern from urinations made in various different places during the patient's daily life, accumulated data from multiple examinations are needed. If there is a portable device, the patient will be able to easily carry it and derive a result by averaging the voiding flow rates and voiding curve patterns accumulated over several days, and present it to the hospital.

Meanwhile, a voiding diary is created as the examinee records their voiding times and voided volumes in a table of certain form made of paper for approximately 3 days. There are various types of voiding diaries depending on the recording method, but among them, the frequency-volume chart (FVC), for recording the voided times and voided volumes, is the most representative form of a voiding diary. The examiner divides the voiding diary that the examinee has written into daytime and nighttime, and derives various voiding diary parameters from the voiding times and voiding volumes, and utilizes the same in diagnosing the examinee. Here, the voiding diary parameters include 24 hours urine volume, nocturnal urine volume, nocturnal polyuria parameter, total number of voids, number of daytime voids, number of nocturnal voids, and functional bladder capacity, etc. In identifying the cause of the symptoms of nocturia, voiding diary is the most important examination as a single unit examination.

However, writing a voiding diary manually causes inconvenience for both the examinee and the examiner. The examinee has to carry a pen and paper all the time and write down every time they urinate, and in order to measure the voided volume, there is also the inconvenience of carrying a measuring cup. Due to such inconvenience, sometimes the examinee fails to accurately record the voided time and voided volume in the voiding diary. Especially, for children and the elderly, it is almost impossible to write done all the records in such voiding diaries. Therefore, in such groups of examinees, only the voiding times are recorded as an expedient method.

Further, the examiner has to quickly analyze the voiding diary recorded by the examinee within the treatment time and derive the voiding diary parameters, but if the changes in the number of voids and voided volumes are extreme due to the examinee's fluid intake and exercise and the like, it is difficult for the examiner to identify the examinee's voiding diary parameters within the limited treatment time. If it becomes possible to automatically record the voiding diary through a portable apparatus, it will be of great help for the examiner to identify the patient's voiding pattern quickly and easily, and find the exact cause of the symptom and treat it.

### SUMMARY

Therefore, a purpose of the present disclosure is to resolve the aforementioned problems of prior art, that is to provide a portable uroflowmetry apparatus, uroflowmetry and creating micturition chart system using the same, and uroflowmetry method, and more particularly, to a portable uroflowmetry apparatus, that allows to easily write voiding information using the portable uroflowmetry apparatus, and to automatically write voiding diary using the portable uroflowmetry apparatus, and uroflowmetry and voiding diary writing system using the same, and uroflowmetry method.

A portable uroflowmetry apparatus according to an embodiment of the present disclosure for achieving the aforementioned purpose may include a tube part where fluid flows; and an examination module that is connected to the tube part, and that measures a flow rate of the introduced fluid over time and analyzes the measured flow rate of the fluid, to derive a result value, wherein the examination module may include an inlet that is connected to one side of the tube part and to which the fluid is introduced, a rotation part that is rotated by the introduced fluid, a controller that measures the flow rate of the introduced fluid over time by analyzing a rotation degree of the rotation part, and analyzes the measured flow rate of the fluid, to derive the result value, and an outlet that is connected to the other side of the tube part and through which the measured fluid is discharged.

Preferably, the controller may include a measurement part that measures the flow rate of the introduced fluid over time, an analyzing part that analyzes the measured flow rate of the fluid, to derive the result value, and a determining part that compares the derived result value with a prestored reference result value, to determine as one of a normal group, low risk group, and high risk group.

Preferably, the result value may include at least one of a maximum flow rate, mean flow rate, voided volume, voiding time and voiding hesitancy time of the fluid, and the examination module may further include a display that displays at least one of an examination date, examination time zone and the result value.

Preferably, the controller may derive a mean result value, that is a mean value of the result value measured for a predetermined period of time, and the display may display the mean result value.

Preferably, the examination module may further include a button for setting an examination time zone so as to differentiate between daytime zone and nighttime zone.

Preferably, the tube part may be formed in a shape that can be reduced in volume or length.

A uroflowmetry system using a portable uroflowmetry apparatus, for achieving the aforementioned purpose may include a portable uroflowmetry apparatus that measures a flow rate of introduced fluid over time, analyzes the measured flow rate of the fluid, to derive a result value, and transmits the derived result value in a wired or wireless manner; and a terminal that receives the result value transmitted from the portable uroflowmetry apparatus, derives a voiding curve pattern based on the received result value, and writes and outputs voiding information based on at least one of the result value and the voiding curve pattern.

Preferably, the result value may include at least one of a maximum flow rate, mean flow rate, voided volume, voiding time and voiding hesitancy time of the fluid, and the voiding information may include at least one of an examination date, examination time zone, the result value and the voiding curve pattern.

Preferably, the terminal may compare the received result value or the voiding curve pattern with a prestored reference result value or a reference voiding curve pattern, and then determine and output as one of a normal group, low risk group and high risk group.

Preferably, the reference voiding curve pattern may include a bell-shaped normal pattern, tower-shaped superflow pattern, compressive pattern, plateau-shaped constrictive pattern, interrupted-shaped pattern and abdominal straining pattern.

Preferably, the terminal may derive a mean result value, that is a mean value of the result value measured for a predetermined period of time, compare the mean result value with a prestored reference result value, and then determine and output as one of a normal group, low risk group, and high risk group.

Preferably, the terminal may derive a mean voiding curve pattern, that is a mean value of the voiding curve pattern measured for a predetermined period of time, and compare the mean voiding curve pattern with a prestored reference voiding curve pattern, and then determine and output as one of a normal group, low risk group, and high risk group.

A voiding diary writing system using a portable uroflowmetry apparatus according to an embodiment of the present disclosure, for achieving the aforementioned purpose may include a portable uroflowmetry apparatus that measures a flow rate of introduced fluid over time, analyzes the measured flow rate of the fluid, to derive a result value, and transmits the derived result value in a wired or wireless manner; and a terminal that receives the result value transmitted from the portable uroflowmetry apparatus, and writes and outputs a voiding diary based on the received result value.

Preferably, the terminal may write the voiding diary that includes at least one of the number of voids by time zone, voided volume by time zone, and urgency degree by time zone.

Preferably, the terminal may write the voiding diary that includes a voiding diary parameter, and the voiding diary parameter may include at least one of a daytime voiding diary parameter, nighttime voiding diary parameter and 24-hour voiding diary parameter.

Preferably, the daytime voiding diary parameter may include at least one of the number of daytime voids, maximum daytime voided volume, mean daytime voided volume and total daytime voided volume.

Preferably, the nighttime voiding diary parameter may include at least one of the number of nocturnal voids, nocturnal voided volume, nocturnal bladder capacity, nocturnal polyuria parameter, and nocturia parameter.

Preferably, the 24-hour voiding diary parameter may include at least one of the number of daily voids, maximum daily voided volume, mean daily voided volume, total daily voided volume, and functional bladder capacity.

Preferably, the terminal may derive a mean result value, that is a mean value of the result value measured for a predetermined period of time, and derive the voiding diary parameter based on the mean result value.

Preferably, the portable uroflowmetry apparatus may include a tube part where fluid flows, and an examination module that is connected to the tube part, and that measures a flow rate of the introduced fluid over time and analyzes the measured flow rate of the fluid, to derive a result value, and transmit the derived result value in a wired or wireless manner, and the examination module may include a first button that can set an examination time zone so as to differentiate between daytime voiding and nocturnal voiding.

Preferably, the examination module may further include a second button for inputting an examination starting time point, and the terminal may compare the examination starting time point input through the second button with a time point when the fluid is introduced into the examination module and started to be measured, to determine as one of a normal, urgent voiding and hesitancy voiding.

A uroflowmetry method according to an embodiment of the present disclosure, for achieving the aforementioned purpose may include an introducing step of introducing fluid into a portable uroflowmetry apparatus; a measuring step of measuring a flow rate of the introduced fluid over time; an analyzing step of analyzing the measured flow rate of the fluid and deriving a result value; a receiving step for a terminal to receive the derived result value; a determining step of deriving a voiding curve pattern based on the received result value, and comparing the result value or the voiding curve pattern with a prestored reference result value or reference voiding curve pattern, and determining as one of a normal group, low risk group, and high risk group; and an outputting step of writing and outputting voiding information based on the result value and the voiding curve pattern, and outputting a result of determination at the determining step.

Preferably, the result value may include at least one of a maximum flow rate, mean flow rate, voided volume, voiding time, and voiding hesitancy time of the fluid, and the voiding information may include at least one of an examination date, examination time zone, the result value and the voiding curve pattern.

Preferably, the reference voiding curve pattern may include a bell-shaped normal pattern, tower-shaped superflow pattern, compressive pattern, plateau-shaped constrictive pattern, interrupted-shaped pattern and abdominal straining pattern.

Preferably, the prestored reference result value or the reference voiding curve pattern may be set differently depending on examinee information, and the examinee information may include information of the examinee's age, gender, examination time zone and disease.

Preferably, the outputting step may output a message that analyzing is impossible when the voided volume is not more than 150 ml.

The present disclosure may provide a portable uroflowmetry apparatus that can continuously measure whether there is an abnormality in the lower urinary tract function and that is easily manageable since it can perform uroflowmetry regardless of location.

The present disclosure may provide a portable uroflowmetry apparatus that allows examining the urine flow of the examinee to derive a quantified result value, thereby allowing the examiner to make a more accurate diagnosis.

The present disclosure may provide a uroflowmetry system and uroflowmetry method, using a portable uroflowmetry apparatus that can continuously measure whether there is an abnormality in the lower urinary tract function and that is easily manageable, since it can perform uroflowmetry regardless of location using the portable uroflowmetry apparatus.

The present disclosure may provide a uroflowmetry system and uroflowmetry method, using a portable uroflowmetry apparatus that allows examining the urine flow of the examinee to derive a quantified result value, thereby allowing the examiner to make a more accurate diagnosis.

The present disclosure may provide a voiding diary writing system, using a portable uroflowmetry apparatus that automatically writes the voiding diary, thereby minimizing the inconvenience of the examinee to handwrite the voiding diary and minimizing the hassle of the examiner to analyze the handwritten voiding diary.

The present disclosure may provide a voiding diary writing system using a portable uroflowmetry apparatus that derives various voiding diary parameters based on the result value received in the portable uroflowmetry apparatus, and writes and outputs the voiding diary that includes the voiding diary parameters, so that the examiner can identify the various voiding diary parameters at a glance, thereby making a more quick and accurate diagnosis on the examinee's symptoms.

### BRIEF DESCRIPTION OF THE DRAWINGS

FIG. 1 is a view illustrating the configuration of a portable uroflowmetry apparatus according to an embodiment of the present disclosure, and uroflowmetry and voiding diary writing system and uroflowmetry method using the same;

FIG. 2 is a view illustrating the configuration of a portable uroflowmetry apparatus of the portable uroflowmetry apparatus according to an embodiment of the present disclosure, and uroflowmetry and voiding diary writing system and uroflowmetry method using the same;

FIGS. 3 and 4 are views illustrating various forms of a lower tube of the portable uroflowmetry apparatus according to an embodiment of the present disclosure, and uroflowmetry and voiding diary writing system and uroflowmetry method using the same;

FIG. 5 is a view illustrating an examination module of the portable uroflowmetry apparatus according to an embodiment of the present disclosure, and uroflowmetry and voiding diary writing system and uroflowmetry method using the same;

FIG. 6 is a view illustrating a side surface of the examination module of the portable uroflowmetry apparatus according to an embodiment of the present disclosure, and uroflowmetry and voiding diary writing system and uroflowmetry method using the same;

FIGS. 7 and 8 are views illustrating an examination module of the portable uroflowmetry apparatus according to an embodiment of the present disclosure, and uroflowmetry and voiding diary writing system and uroflowmetry method using the same, with a housing part removed;

FIGS. 9 and 10 are views illustrating a front view of the examination module of the portable uroflowmetry apparatus according to an embodiment of the present disclosure, and uroflowmetry and voiding diary writing system and uroflowmetry method using the same, with a housing part removed;

FIG. 11 is a view illustrating the connection between components of the examination module of the portable uroflowmetry apparatus according to an embodiment of the present disclosure, and uroflowmetry and voiding diary writing system and uroflowmetry method using the same;

FIG. 12 is a view illustrating a voiding diary being written in a terminal of the portable uroflowmetry apparatus according to an embodiment of the present disclosure, and uroflowmetry and voiding diary writing system and uroflowmetry method using the same;

FIG. 13 is a view illustrating a voiding diary that includes voiding diary parameters, being written in the terminal of the portable uroflowmetry apparatus according to an embodiment of the present disclosure, and uroflowmetry and voiding diary writing system and uroflowmetry method using the same;

FIG. 14 illustrates a voiding curve pattern for a predetermined period of time of the examinee and mean value thereof, derived by the terminal of the portable uroflowmetry apparatus according to an embodiment of the present disclosure, and uroflowmetry and voiding diary writing system and uroflowmetry method using the same;

FIG. 15 is a view illustrating a bell-shaped normal pattern of a reference voiding curve pattern of the portable uroflowmetry apparatus according to an embodiment of the present disclosure, and uroflowmetry and voiding diary writing system and uroflowmetry method using the same;

FIG. 16 is a view illustrating a tower-shaped superflow pattern of the reference voiding curve pattern of the portable uroflowmetry apparatus according to an embodiment of the present disclosure, and uroflowmetry and voiding diary writing system and uroflowmetry method using the same;

FIG. 17 is a view illustrating a compressive pattern of the reference voiding curve pattern of the portable uroflowmetry apparatus according to an embodiment of the present disclosure, and uroflowmetry and voiding diary writing system and uroflowmetry method using the same;

FIG. 18 is a view illustrating a plateau-shaped constrictive pattern of the reference voiding curve pattern of the portable uroflowmetry apparatus according to an embodiment of the present disclosure, and uroflowmetry and voiding diary writing system and uroflowmetry method using the same;

FIG. 19 is a view illustrating an interrupted-shaped pattern of the reference voiding curve pattern of the portable uroflowmetry apparatus according to an embodiment of the present disclosure, and a uroflowmetry and voiding diary writing system and uroflowmetry method using the same;

FIG. 20 is a view illustrating an abdominal straining pattern of the reference voiding curve pattern of the portable uroflowmetry apparatus according to an embodiment of the present disclosure, and uroflowmetry and voiding diary writing system and uroflowmetry method using the same;

FIGS. 21 to 23 are views illustrating various reference voiding curve patterns of the portable uroflowmetry apparatus according to an embodiment of the present disclosure, and uroflowmetry and voiding diary writing system and uroflowmetry method using the same;

FIG. 24 is a view illustrating a voiding diary actually written in the terminal of the portable uroflowmetry apparatus according to an embodiment of the present disclosure, and uroflowmetry and voiding diary writing system and uroflowmetry method using the same;

FIG. 25 is a view illustrating an actual voiding curve pattern included in the voiding diary being written in the terminal of a portable uroflowmetry apparatus according to an embodiment of the present disclosure, and uroflowmetry and voiding diary writing system and uroflowmetry method using the same; and

FIG. 26 is a flowchart of the uroflowmetry method according to an embodiment of the present disclosure.

### DETAILED DESCRIPTION

Hereinbelow, some embodiments of the present disclosure will be described in detail through the exemplary drawings. In adding reference numerals to components of each drawing, it should be noted that even if the components are displayed on different drawings, like reference numerals are used for like components as much as possible.

Further, in describing the embodiments of the present disclosure, if it is determined that a specific description of a related well-known configuration or a function interrupts the understanding of the embodiments of the present disclosure, detailed description thereof will be omitted.

Further, in describing the components of the present disclosure, terms such as a first, a second, A, B, (a), (b) and the like may be used. Such terms are merely used to distinguish those components from other components, and such terms do not limit the nature, sequence or order of the corresponding components.

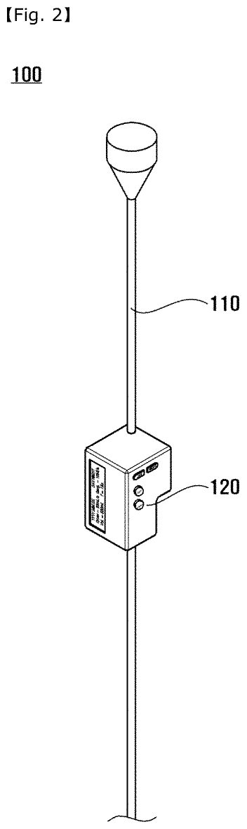

Referring to FIG. 2, the configuration and operations of a portable uroflowmetry apparatus 100 according to an embodiment of the present disclosure will be described hereinbelow.

FIG. 2 is a view illustrating the configuration of a portable uroflowmetry apparatus of the portable uroflowmetry apparatus according to an embodiment of the present disclosure, and uroflowmetry and voiding diary writing system and uroflowmetry method using the same.

The portable uroflowmetry apparatus 100 is configured to measure a flow rate of an introduced fluid over time, analyze the measured flow rate of the fluid, to derive a result value, and transmit the derived result value in a wired or wireless manner. The portable uroflowmetry apparatus 100 is connected with a terminal 200 to be described later, in a wired or wireless manner.

Referring to FIG. 2, the portable uroflowmetry apparatus 100 may include a tube part 110 and an examination module 120.

The tube part 110 is where fluid flows. The tube part 110 is connected and communicates with the examination module 120 to be described later.

In the present disclosure, the fluid refers to the examinee's urine being introduced into the tube part 110. The present disclosure measures the flow rate over time of the introduced urine of the examinee, thereby deriving a result value for diagnosing a dysfunction of the lower urinary tract.

In more detail, the tube part 110 may include an input part 111, an upper tube 112, and a lower tube 113.

The input part 111 is where the fluid is initially introduced. An upper portion of the input part 111 is open, and the input part 111 has a shape of a funnel, that is, of which the diameter of a lower portion is smaller than that of the upper portion, and the lower portion is connected and communicates with the upper tube 112.

The upper tube 112 has a path formed such that the fluid introduced from the input part 111 flows to an inlet (i) of the examination module 120 to be described later. One side of the upper tube 112 is connected and communicates with the inlet (i) of the examination module 120 to be described later.

The lower tube 113 has a path formed such that the fluid measured in the examination module 120 to be described later can be discharged to outside. One side of the lower tube 113 is connected and communicates with an outlet (o) of the examination module 120 to be described later.

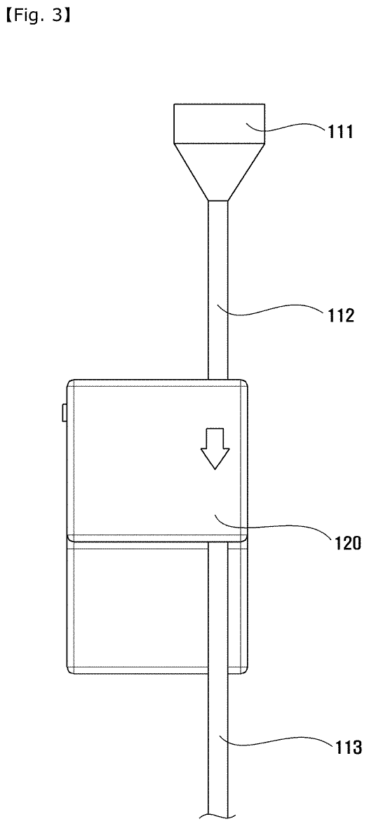

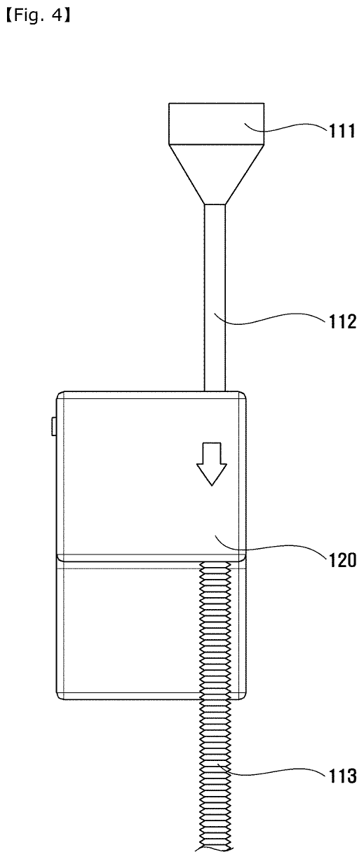

FIGS. 3 and 4 are views illustrating various forms of the lower tube of the portable uroflowmetry apparatus according to an embodiment of the present disclosure, and uroflowmetry and voiding diary writing system and uroflowmetry method using the same.

Referring to FIGS. 3 and 4, in one embodiment, the upper tube 112 or the lower tube 113 may be made of multiple components and have a slidable form of which the volume or length can be reduced so that the examinee can easily carry and measure the urine flow. Further, in one embodiment, the upper tube 112 or the lower tube 113 may have a bellows shape so that the volume or length can be reduced.

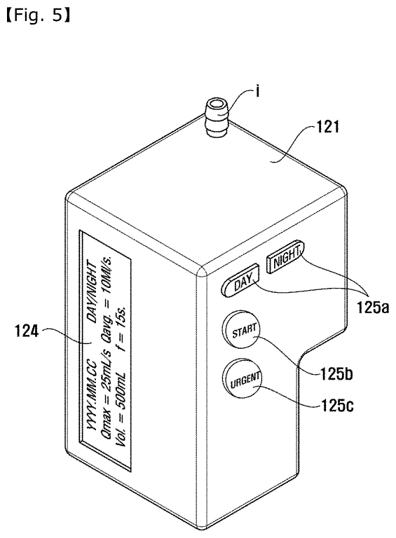

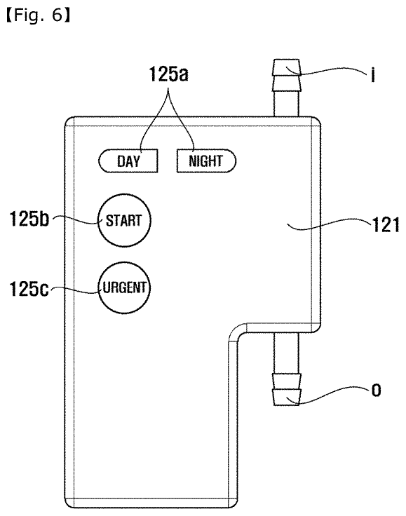

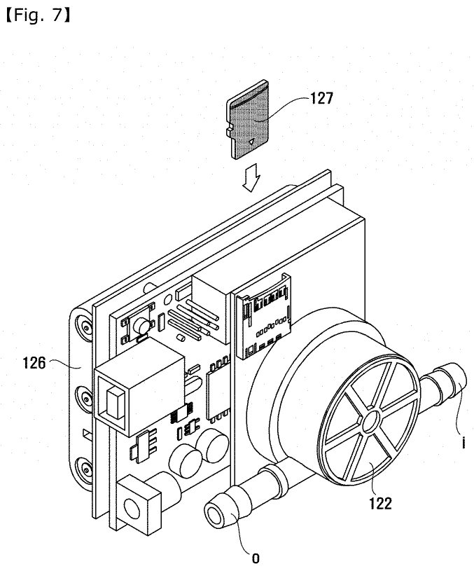

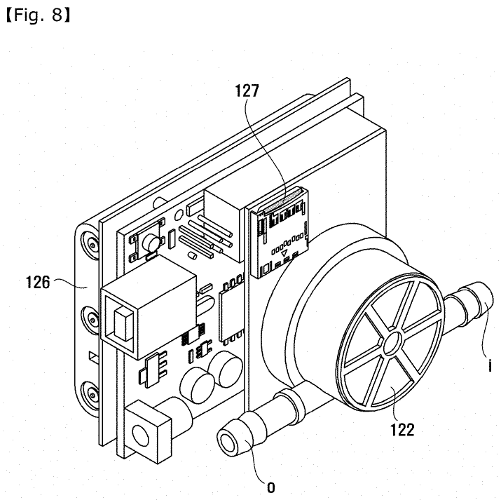

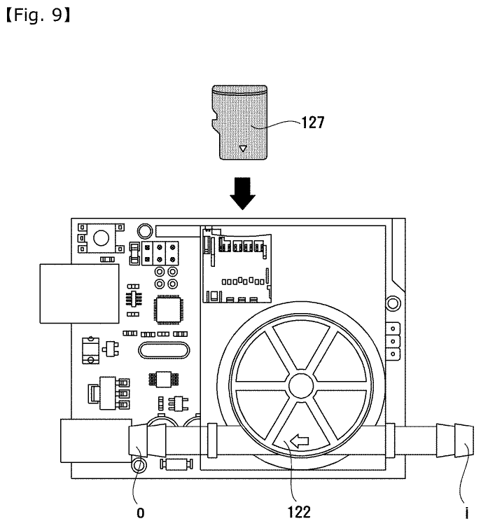

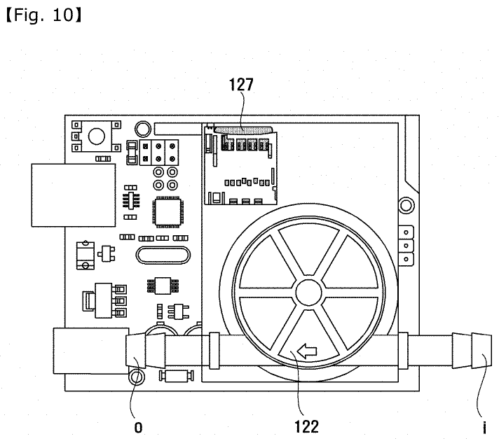

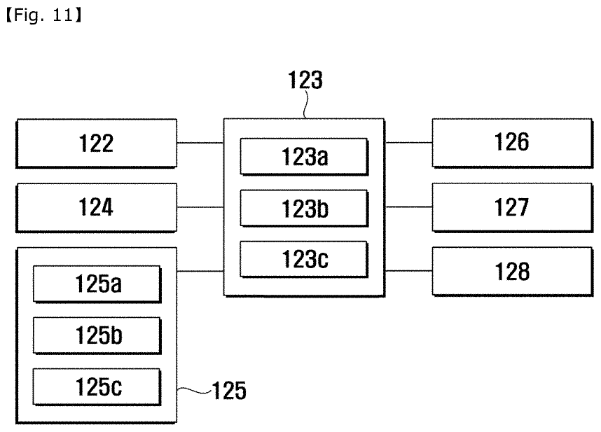

FIG. 5 is a view illustrating an examination module of the portable uroflowmetry apparatus according to an embodiment of the present disclosure, and uroflowmetry and voiding diary writing system and uroflowmetry method using the same; FIG. 6 is a view illustrating a side surface of the examination module of the portable uroflowmetry apparatus according to an embodiment of the present disclosure, and uroflowmetry and voiding diary writing system and uroflowmetry method using the same; FIGS. 7 and 8 are views illustrating an examination module of the portable uroflowmetry apparatus according to an embodiment of the present disclosure, and uroflowmetry and voiding diary writing system and uroflowmetry method using the same, with a housing part removed; FIGS. 9 and 10 are views illustrating a front view of the examination module of the portable uroflowmetry apparatus according to an embodiment of the present disclosure, and uroflowmetry and voiding diary writing system and uroflowmetry method using the same, with a housing part removed; and FIG. 11 is a view illustrating the connection between components of the examination module of the portable uroflowmetry apparatus according to an embodiment of the present disclosure, and uroflowmetry and voiding diary writing system and uroflowmetry method using the same.

Referring to FIGS. 5 to 11, the examination module 120 of the portable uroflowmetry apparatus 100 is for measuring the flow rate of the introduced fluid over time, analyzing the measured flow rate of the urine and deriving a result value, and transmitting the derived result value in a wired or wireless manner. Such an examination module 120 of the portable uroflowmetry apparatus 100 may be connected with and communicate with the aforementioned tube part 110.

In more detail, the examination module 120 may include a housing part 121, an inlet (i), a rotation part 122, a controller 123, an outlet (o), a display 124, a button 125, a power supply 126, a storage 127 and a communicator 128.

The housing part 121 is what forms an exterior of the examination module 120. The housing part 121 is formed to have a portable size, and has the display 124 and the button 125 installed at each side. Inside of the housing part 121, an installation space is provided, where the inlet (i), the rotation part 122, the controller 123, the outlet (o), the power supply 126, the storage 127 and the communicator 128, to be described later, may be installed.

The inlet (i) is what forms a path so that fluid can be introduced into the rotation part 122 to be described later. The inlet (i) is installed on the housing part 121 such that a portion of the inlet (i) is exposed, and is connected to one side of the upper tube 112.

The rotation part 122 is configured to be rotated by the fluid introduced through the inlet (i), and is electrically connected to a measurement part 123a to be described later.

In one embodiment, the rotation part 122 may be formed as an impeller having a shape where a plurality of wing plates are radially installed between one pair of circular plates, and the rotation part 122 may be rotated in one direction around a rotation shaft by the load of the fluid.

The controller 123 may be connected to the rotation part 122 to measure the flow rate of the fluid over time, and then analyze the measurement to derive the result value. Further, the controller 123 controls the display 124, the button 125, the power supply 126, the storage 127 and the communicator 128, and the controller 123 is electrically connected to the rotation part 122, the display 124, the button 125, the power supply 126, the storage 127 and the communicator 128. In one embodiment, the controller 123 may be made in a microcontroller unit (MCU) in which an arithmetic processor, a memory and the like are embedded in one integrated circuit (IC), and can perform both operations and storage functions.

In more detail, the controller 123 may include a measurement part 123a, an analyzing part 123b and a determining part 123c. In one embodiment, in analyzing the measured flow rate of the fluid or the flow rate of the fluid, the determining part 123c may be implemented through a hardware having a semiconductor chip form, that can process mathematical operations of algorithms.

The measurement part 123a is for analyzing the degree of rotation motion of the rotation part 122, to measure the flow rate of the introduced fluid over time. The measurement part 123a is electrically connected to the analyzing part 123b to be described later.

In more detail, flow rate refers to the ratio of volume or mass of the fluid flowing through an arbitrary cross-section to time, and in the present disclosure, it uses the fact that in a case where, by the examinee's urination, the fluid passes through the tube part 110 having an arbitrary cross-section and is introduced into the examination module 120, the rotation speed of the rotation part 122 is proportionate to the flow rate. That is, the measurement part 123a of the present disclosure can measure the flow rate of the introduced fluid over time through the Equation 1 below.

Q=Kω (1)

Here, Q refers to the flow rate of the fluid, K refers to the cross-sectional area of the tube part, and ω refers to the rotation speed of the rotation part.

In one embodiment, the measurement part 123a may be provided as a tachometer that is connected to a rotation shaft of the rotation part 122 to make a pulse signal for a rotation motion of the rotation shaft, or a rotary encoder connected to the rotation shaft to digitally signal the rotation motion of the rotation shaft, but there is no limitation thereto, and thus any configuration that may be connected to the rotation shaft of the rotation part 122 to measure the rotation motion of the rotation shaft and to electrically signal the same may be used as the measurement part 123a.

The analyzing part 123b is for analyzing the flow rate of the introduced fluid over time, measured in the measurement part 123a, to derive the result value, and the analyzing part 123b is electrically connected to the determining part 123c.

Here, the result values derived in the analyzing part 123b include a maximum flow rate (Qmax), a mean flow rate, a voided volume, a voiding time and hesitancy time, by time zone. Here, the maximum flow rate refers to the maximum value of the speed of the voided fluid, the mean flow rate refers to the mean value of the speed of the voided fluid, the voided volume refers to the total amount of the voided fluid, the voiding time refers to the total time it took for the examinee in voiding, and the hesitancy time refers to the time the examinee hesitated before starting to void.

In one embodiment, the analyzing part 123b may derive a mean result value, that is a mean value of the result values measured for a predetermined period of time, for example, for 3 days. Generally, the examiner lets the examinee record the parameters such as the voided volume for 3 days, and then determines the symptoms of the examinee based on the record. However, in this method, there is a hassle that the examinee has to record every urination, and the examiner has the inconvenience of separately calculating the 3 days' result values. Accordingly, the analyzing part 123b may analyze the flow rate of the fluid over time, measured for the predetermined period of time, and derive the result values of the maximum flow rate, mean flow rate, voided volume, voiding time and hesitancy time, respectively, and then average the result values to derive the mean result value. However, the mean result value may also be derived by the terminal 200 to be described later.

The determining part 123c may compare the result value derived in the analyzing part 123b with a prestored reference result value, and determine as one of a normal group, low risk group, and high risk group. The determining part 123c is electrically connected to the analyzing part 123b.

Here, the prestored reference result values refer to the maximum flow rate, mean flow rate, voided volume, voiding time and hesitancy time observed in a normal case, and the maximum flow rate, mean flow rate, voided volume, voiding time and hesitancy time observed in an abnormal case (preferably per each disease).

In one embodiment, examinee information may include information of examinee's age, examinee's gender, examination time and disease (prostatic hyperplasia, urinary incontinence, nocturia, overactive bladder symptoms etc.), and the prestored reference result values to be compared in the determining part 123c may be set differently depending on the aforementioned examinee information. That is, upon comparing the result values derived in the analyzing part 123b, a look up table for classifying the examinee into one of the normal group, low risk group and high risk group may be verified according to the aforementioned examinee information. Accordingly, as the examinee information is reflected when performing the uroflowmetry, there is an effect that a more accurate identification is possible.

The outlet (o) is where a path is formed so that the fluid measured in the measurement part 123a can be discharged outside. The outlet (o) is installed on the housing part 121 such that a portion of the outlet (o) is exposed, and is connected to one side of the lower tube 113.

The display 124 is for displaying at least one or more of the examination date, examination time, result value derived in the analyzing part 12b and the result determined in the determining part 123c, and is installed at one side of the housing part 121. That is, the display 124 may receive the examination date, examination time and result value derived in the analyzing part 123b, and display the same, and may also output the result determined in the determining part 123c together.

In one embodiment, depending on the result value being displayed on the display 124, or whether the determined result corresponds to the normal group, low risk group, or high risk group, the result value or the determined result may be displayed on the display 124 in different colors (for example, green, orange, red, etc.)

In one embodiment, at one side end of the housing part 121, a light source for alarm may be installed, and here, depending on the result value being displayed on the display 124, and whether the determined result corresponds to the normal group, low risk group, or high risk group, different colors may be emitted from the light source (for example, green, orange, red, etc.)

In one embodiment, since when the voided volume of the examinee is not more than 150 ml, it is difficult to perform an accurate measurement, the display 124 may output a message saying that analyzing is impossible when the voided volume is not more than 150 ml, and a voice may be accompanied as well.

The button 125 is configured for the examinee to set the examination time, input the time point of starting the examination and input the urgent degree of urgent voiding. In more detail, the button 125 may include a first button 125a, a second button 125b, and a third button 125c.

The first button 125a is configured for the examinee to set the examination time zone, that is, to differentiate between daytime and nighttime of when the examination was performed so that the examinee can differentiate between daytime voiding and nighttime voiding. The first button 125a may be installed at one side of the housing part 121. When the examinee operates the first button 125a, the corresponding time zone (whether it is daytime zone or nighttime zone) in which the examinee performed the uroflowmetry is stored accordingly, and the corresponding time zone is output on the display 124.

The second button 125b is configured for the examinee to input the time point of starting the examination. The second button 125b may be installed at one side of the housing part 121. When the examinee presses the second button 125b, the time point of starting the examination is stored, and thereafter, when fluid is introduced into the examination module 120 through the tube part 110 and the rotation part 122 is rotated, the time point when the fluid started to be measured is stored, and the time point of starting the examination and the time point of measuring the fluid are included in the result value and transmitted to the terminal 200 to be described later. Then, the terminal 200 that received the result value compares the time point of starting the examination input through the second button 125b and the time point when the fluid was introduced into the examination module 120 and started to be measured, and thus determines as one of a normal voiding, urgent voiding and hesitant voiding.

In more detail, urgent voiding refers to a state where one cannot hold the urine and has to void immediately, whereas hesitant voiding refers to a state where one cannot void immediately but hesitates. The terminal 200 may determine the voiding as urgent voiding when the interval between the time point of starting examination and the time point of measuring the fluid is not more than a predetermined interval (for example, not more than 5 seconds), and determine the voiding as hesitant voiding when the interval between the time point of starting examination and the time point of measuring the fluid is not less than a predetermined interval (for example, 30 seconds), and determine the voiding as normal voiding, that is neither urgent voiding nor hesitant voiding when the interval between the time point of starting examination and the time point of measuring the fluid is within a predetermined interval (for example, not less than 5 seconds and not more than 30 seconds).

The third button 125c is configured for the examinee to input the urgent degree of urgent voiding. The third button 125c may be installed at one side of the housing part 121. The urgent degree of urgent voiding may be included and stored in the result value, and the terminal 200 to be described later may receive the urgent degree of urgent voiding included and stored in the result value. The terminal 200 may use this result value to write a voiding diary where the urgent degree of urgent voiding over time is included. Here, the urgent degree of urgent voiding is a parameter indicating how much urgent the voiding was at the time of voiding of the examinee, and the urgent degree of urgent voiding may be classified into, for example, 4 stages (stage 1: not urgent, stage 4: very urgent, etc.). Here, the third button 125c is provided in a button form so that as it is pressed by the examinee by a predetermined number of times, each stage may be displayed on the display 124 (for example, when pressed once, stage 1 may be displayed, when pressed 4 times, stage 4 may be displayed, and when pressed 5 times, stage 1 may be displayed again) and stored.

In one embodiment, the aforementioned first button 125a, the second button 125b or the third button 125c may be formed in one area of the display 124, and may perform their respective functions by a touch method.

The power source 126 is for supplying power to the examination module 120. The power source 126 may be connected to an external power source (for example, 220V), to supply power to the examination module 120, or the power source 126 may be provided as a battery to supply power to the examination module 120. When the power source 126 is provided as a battery, in the housing part 121, a cover may be formed, for opening and closing a portion in which the battery may be mounted. The cover may be provided in various manner such as attach/detach type cover, slide type cover, and bolt type cover, etc.

The storage 127 is for storing the result value derived in the analyzing part 123b and the reference result value, and for storing the result determined in the determining part 123c. The storage part 127 may be electrically connected to the controller 123.

In one embodiment, the storage 127 may be provided as a mini SD card, micro SD card, multimedia card (MMC), memory stick (MS, MSD, MSPD), and compact flash memory card (CF), but there is no limitation thereto, and thus any configuration may be used as long as it performs the function of storing data.

The communicator 128 is for communicating in a wireless manner using a network or in a wired method using the terminal 200 and USB port, and through this, the result value derived in the analyzing part 123b and the result determined in the determining part 123c may be transmitted to the terminal 200 to be described later in a wired or wireless manner. Further, the reference result value may be stored in the terminal 200, and the communicator 128 may receive such a reference result value.

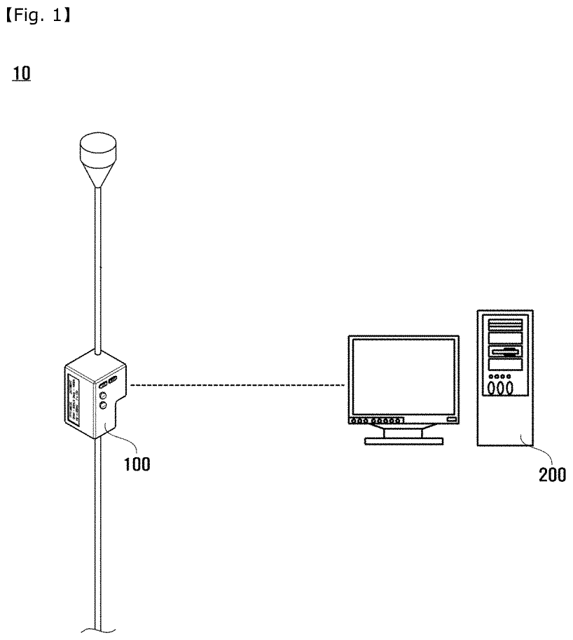

Referring to FIG. 1, the configuration and operations of a uroflowmetry and voiding diary writing system using a portable uroflowmetry apparatus 10 according to an embodiment of the present disclosure will be described hereinbelow.

FIG. 1 is a view illustrating the configuration of a portable uroflowmetry apparatus according to an embodiment of the present disclosure, and uroflowmetry and voiding diary writing system and uroflowmetry method using the same.

Referring to FIG. 1, the uroflowmetry and voiding diary writing system using a portable uroflowmetry apparatus 10 according to an embodiment of the present disclosure may include a portable uroflowmetry apparatus 100 and a terminal 200.

Here, the portable uroflowmetry apparatus 100 has the same configuration as aforementioned, and thus repeated description will be omitted.

The terminal 200 is for receiving the result value transmitted from the portable uroflowmetry apparatus 100, and for writing the voiding information and/or voiding diary based on the received result value and outputting the same. The terminal 200 is connected with the aforementioned portable uroflowmetry apparatus 100 in a wired or wireless manner.

In one embodiment, the terminal 200 may include a general use PC such as a notebook, desktop, laptop equipped with a WEB browser, and as a wireless communication device guaranteeing portability and mobility, may include all kinds of handheld based wireless communication apparatus such as terminals supporting PCS(personal communication system), GSM(global system for mobile communications), PDC(personal digital cellular), PHS(personal handyphone system), PDA(personal digital assistant), CDMA(code division multiple access)-2000, W-CDMA(wcode division multiple access), Wi-Fi(wireless LAN, WLAN), WiBro(wireless broadband), WiMAX(world interoperability for mocrowave access), HSDPA(high speed downlink packet access), HSUPA(high speed uplink packet access), HSPA(high speed packet access), and LTE(long term evolution).

In one embodiment, the terminal 200 may include one or more ports for receiving various terminals such as a processor such as a microprocessor for operating/adjusting other components of the terminal, a user interface for receiving an input control signal and data from a user of the terminal 200, a memory for storing data, a display for displaying various images, a communication unit for data communicating with various external devices such as a portable uroflowmetry apparatus, a power source cable, and a USB, etc.

In one embodiment, the voiding information being written and output in the terminal 200 may include at least one of the result values such as the examination date and examination time zone, maximum flow rate of the fluid, mean flow rate of the fluid, voided volume, voiding time and voiding hesitancy time.

In one embodiment, the terminal 200 may compare the result value received in the portable uroflowmetry apparatus 100 with the prestored reference result value, and determine as one of the normal group, low risk group, and high risk group, and output the same.

Here, the prestored reference result value refers to the maximum flow rate, mean flow rate, voided volume, voiding time and voiding hesitancy time observed in a normal case, and the maximum flow rate, mean flow rate, voided volume, voiding time and voiding hesitancy time observed in an abnormal case (preferably for each disease).

In one embodiment, the examinee information may include information of examinee's age, examinee's gender, examination time zone and disease (prostatic hyperplasia, urinary incontinence, nocturia, overactive bladder symptoms etc.), and the prestored reference result values or the reference voiding curve pattern to be compared in the terminal 200 may be set differently depending on the aforementioned examinee information. That is, upon comparing the result values derived in the portable uroflowmetry apparatus 100, a look up table for classifying the examinee into one of the normal group, low risk group and high risk group may be verified according to the aforementioned examinee information. Accordingly, as the examinee information is reflected when performing the uroflowmetry, there is an effect that a more accurate identification is possible.

In another embodiment, the terminal 200 may use the received result value to derive a graph regarding the voiding curve pattern of the examinee. Here, the voiding information being written and output in the terminal 200 may include the voiding curve pattern.

Further, the terminal 200 may compare the derived voiding curve pattern of the examinee with the prestored reference voiding curve pattern, and determine as one of the normal group, low risk group and high risk group, and output the same.

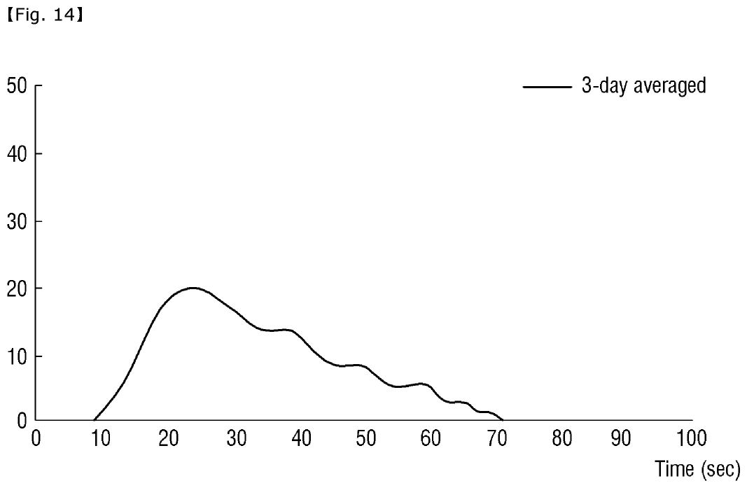

FIG. 14 illustrates the voiding curve pattern for a predetermined period of time of the examinee and mean value thereof, derived by the terminal of the portable uroflowmetry apparatus according to an embodiment of the present disclosure, and uroflowmetry and voiding diary writing system and uroflowmetry method using the same.

Referring to FIG. 14, the terminal 200 may derive a mean voiding curve pattern by deriving a graph regarding the voiding curve pattern of the examinee for a predetermined period of time, for example, 3 days, respectively, using the result values measured for those 3 days, and then averaging the same. Thereafter, the terminal 200 may compare the derived mean voiding curve pattern with the prestored reference voiding curve pattern, to determine as one of the normal group, low risk group and high risk group, and the examiner may use the derived mean voiding curve pattern in diagnosing the symptoms.

Here, the reference voiding curve patterns that may be prestored in the terminal 200 include bell-shaped normal pattern, tower-shaped superflow pattern, compressive pattern, plateau-shaped constrictive pattern, interrupted-shaped pattern and abdominal straining pattern.

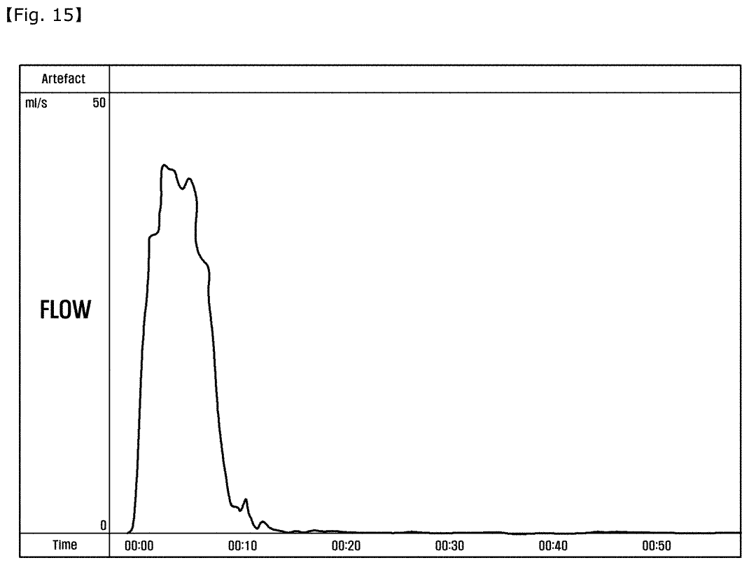

FIG. 15 is a view illustrating a bell-shaped normal pattern of the reference voiding curve pattern of the portable uroflowmetry apparatus according to an embodiment of the present disclosure, and uroflowmetry and voiding diary writing system and uroflowmetry method using the same.

Referring to FIG. 15, the bell-shaped normal pattern is a bell-shaped voiding curve pattern that is derived when there is no dysfunction of lower urinary tract of the examinee and the flow peak of urine flow is appropriate. The bell-shaped normal pattern is a voiding curve pattern of normal range where neither voiding hesitancy nor terminal dribble is observed.

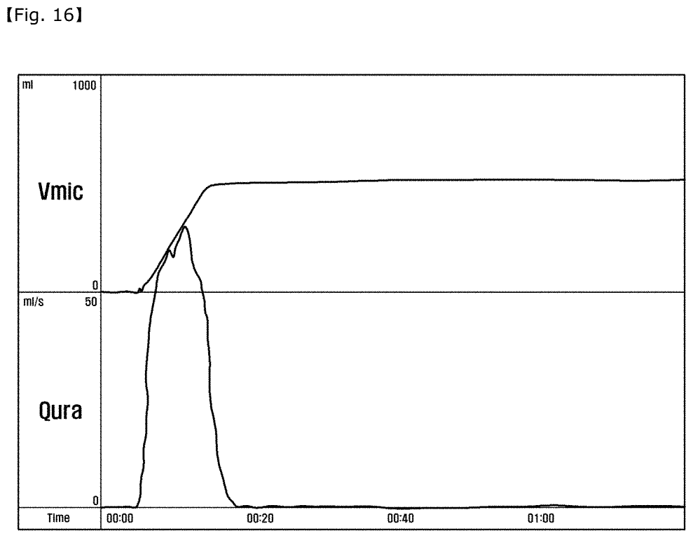

FIG. 16 is a view illustrating a tower-shaped superflow pattern of the reference voiding curve pattern of the portable uroflowmetry apparatus according to an embodiment of the present disclosure, and uroflowmetry and voiding diary writing system and uroflowmetry method using the same.

Referring to FIG. 16, the tower-shaped superflow pattern refers to a voiding curve pattern that is similar to the bell-shaped normal pattern, but the peak value of the urine flow is higher than that of the normal range. Such a tower-shaped superflow pattern is caused when there is detrusor overactivity of the examinee, or when urethral resistance is very low.

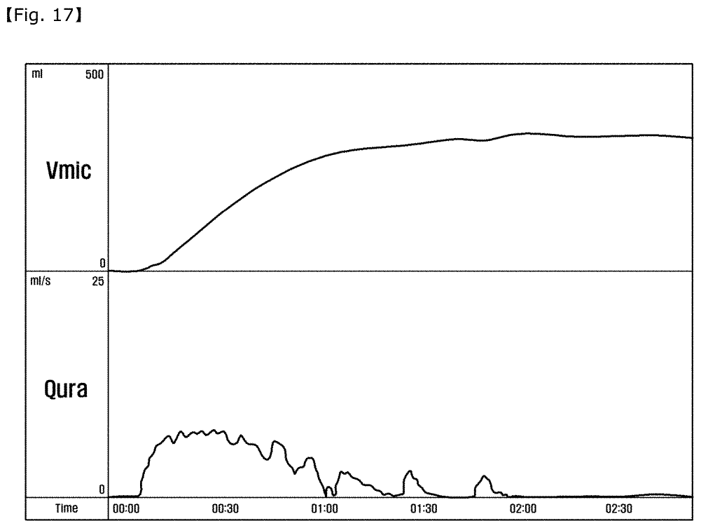

FIG. 17 is a view illustrating a compressive pattern of the reference voiding curve pattern of the portable uroflowmetry apparatus according to an embodiment of the present disclosure, and uroflowmetry and voiding diary writing system and uroflowmetry method using the same.

Referring to FIG. 17, the compressive pattern is a pattern that is commonly observed in examinees with benign prostatic hyperplasia (BPH). When bladder contraction pressure increases during urination, the urethral aperture expands. In benign prostatic hyperplasia (BPH), the enlarged prostate presses on the urethra and limits the urethral aperture, so the compressive pattern refers to a voiding curve pattern in which voiding cannot be performed normally. In such symptoms, although the diameter of the urethra is limited, there is room for small dilation during urination, and thus intermittency or terminal dribble may be observed in the last part of the voiding curve pattern.

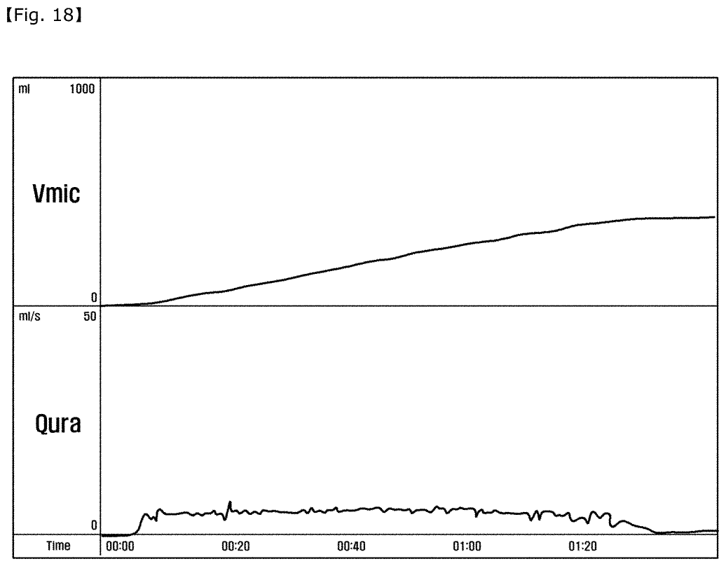

FIG. 18 is a view illustrating a plateau-shaped constrictive pattern of the reference voiding curve pattern of the portable uroflowmetry apparatus according to an embodiment of the present disclosure, and uroflowmetry and voiding diary writing system and uroflowmetry method using the same.

Referring to FIG. 18, the plateau-shaped constrictive pattern refers to a voiding curve pattern in which the peak of the urine flow is not properly formed, that is, the peak is maintained flat. The plateau-shaped constrictive pattern means that the urethral aperture is never dilated beyond a certain level, and this voiding curve pattern is observed in cases of urethral stricture.

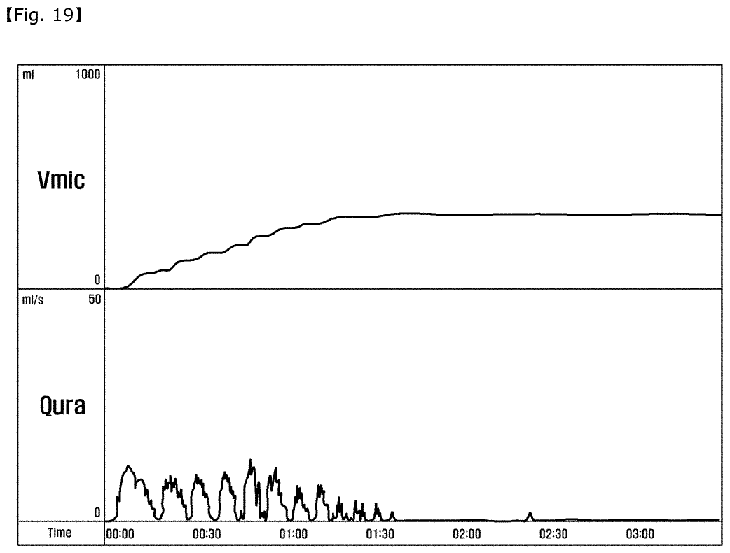

FIG. 19 is a view illustrating an interrupted-shaped pattern of the reference voiding curve pattern of the portable uroflowmetry apparatus according to an embodiment of the present disclosure, and a uroflowmetry and voiding diary writing system and uroflowmetry method using the same.

Referring to FIG. 19, the interrupted-shaped pattern refers to a voiding curve pattern in which the urine flow is interrupted multiple times, which is commonly observed in cases where voiding is performed by abdominal pressure, and it means that a long-term follow-up examination of the upper urinary tract is required.

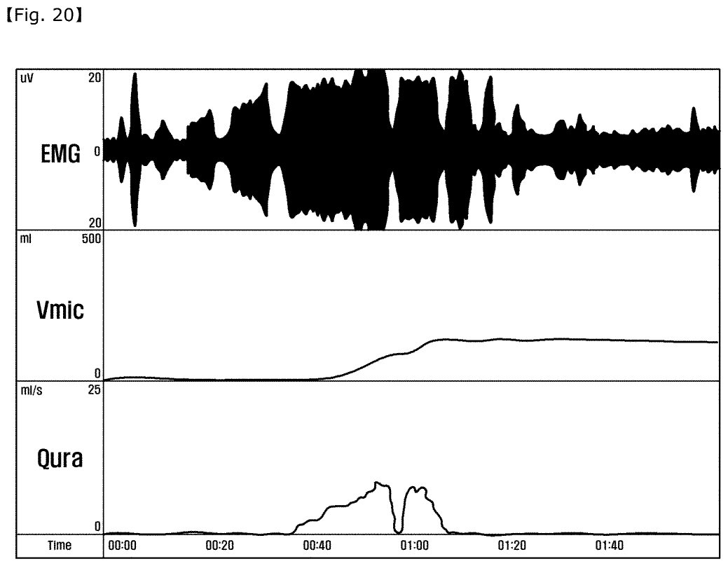

FIG. 20 is a view illustrating an abdominal straining pattern of the reference voiding curve pattern of the portable uroflowmetry apparatus according to an embodiment of the present disclosure, and uroflowmetry and voiding diary writing system and uroflowmetry method using the same.

Referring to FIG. 20, the abdominal staining pattern refers to a voiding curve pattern observed when voiding is performed by abdominal pressure, and is observed in cases where there is no bladder contractility and very low urethral resistance. Here, if a flow-EMG study using an EMG patch is performed together with the uroflowmetry using the portable uroflowmetry apparatus 100 of the present disclosure, it can be helpful to accurately distinguish whether the examinee's symptoms are due to abdominal pressure urination.

Meanwhile, wag artifact is an artifact that occurs during the uroflowmetry process, that is, in the process of deriving a voiding curve pattern, and is caused by a collection of urine streams on the outer side of the uroflowmetry apparatus. Since this wag artifact is observed in the peak part of a voiding curve pattern, it is desirable to correct it to determine the maximum flow rate (Qmax) by averaging with peak surroundings. In addition, artificial noise signal errors refer to signal errors caused by minor collisions such as unintentional kicking of a sensor-equipped examination equipment, so it is necessary to correct these errors.

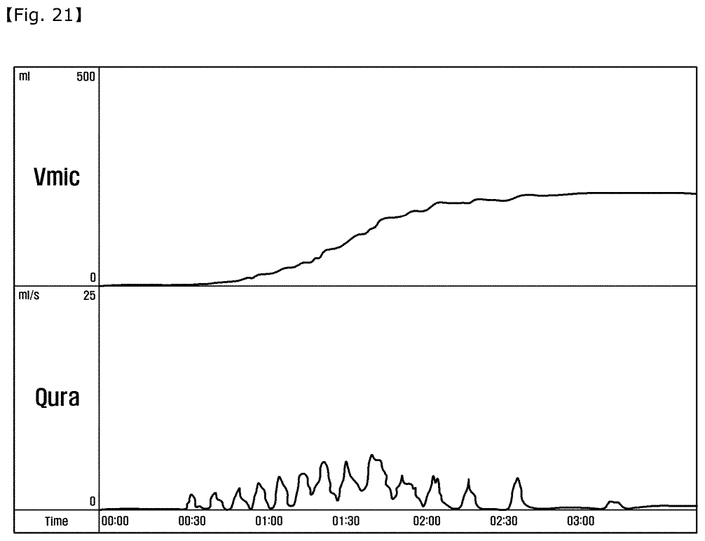

FIGS. 21 to 23 are views illustrating various reference voiding curve patterns of the portable uroflowmetry apparatus according to an embodiment of the present disclosure, and uroflowmetry and voiding diary writing system and uroflowmetry method using the same.

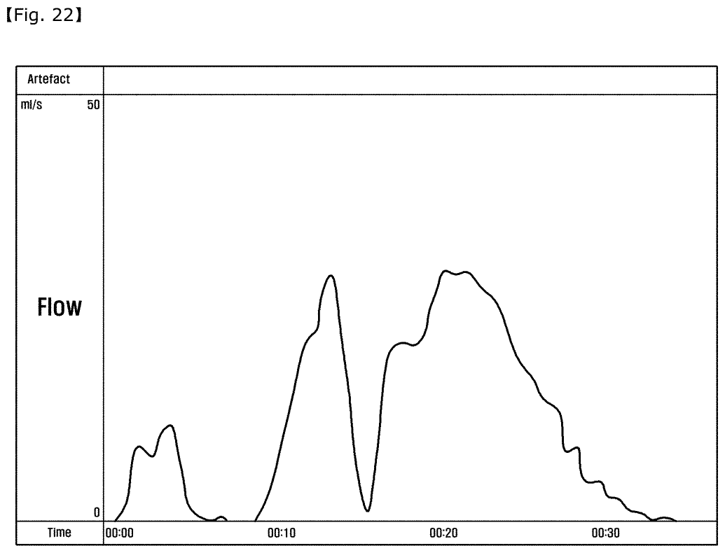

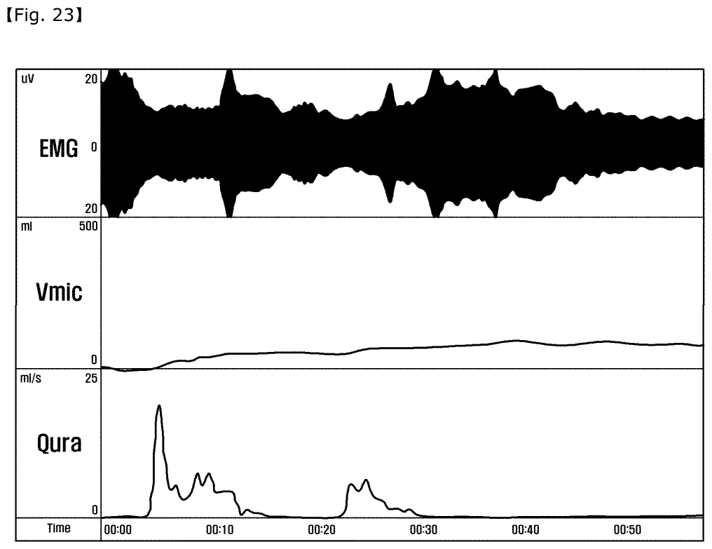

Referring to FIGS. 21 to 23, besides the aforementioned voiding curve patterns, the reference voiding curve patterns that may be prestored in the terminal 200 may include various abnormal voiding curve patterns, such as the intermittent straining flow pattern of FIG. 21, the staccato-shaped pattern in the dysfunctional voiding of FIG. 22, and the detrusor-striated sphincter dyssynergia (DSD) of FIG. 23.

In another embodiment, the terminal 200 may determine the examinee as one of the normal group, low risk group and high risk group, using a pre-learned artificial neural network.

In more detail, the artificial neural network of the terminal 200 may sustainably learn the reference result value and reference voiding curve pattern (preferably, the result value and voiding curve pattern derived from actual disease cases), so that when the portable uroflowmetry apparatus 100 transmits the derived result value of the examinee to the terminal 200, the terminal 200 may determine the examinee as one of the normal group, low risk group and high risk group using the pre-learned artificial neural network, and include such result of determination in the voiding diary. Meanwhile, here the artificial neural network of the terminal 200 may be provided as a deep convolutional neural network (DCNN), but there is no limitation thereto.

When determining through the maximum flow rate in the case of, for example, the benign prostatic hyperplasia (BPH) as in Table 1 below, when the maximum flow rate (Qmax) is not less than 15.1 ml/s as in Example 1, the terminal 200 may determine the examinee as normal, and when the Qmax is between 12.1 ml/s˜15.0 ml/s as in Example 2, the terminal 200 may determine the examinee as in the low risk group (that is, patient that needs to be managed), and when the Qmax is not more than 12.0 ml/s as in Examples 3 and 4, the terminal 200 may determine the examinee as in the high risk group (that is, patient that needs hospital treatment). In addition, when determining whether the examinee is in the risk group through the learned artificial neural network, in the case of Example 3, the terminal 200 may determine in more detail that there are abnormal findings and that medical treatment is required, and in the case of Example 4, the terminal 200 may determine in more detail that there are abnormal findings caused by urethral stricture and that medical treatment is required.

Here, the prestored reference result values refer to the maximum flow rate, mean flow rate, voided volume, voiding time and voiding hesitancy time observed in normal cases, and the maximum flow rate, mean flow rate, voided volume, voiding time and voiding hesitancy time observed in abnormal cases (preferably, for each disease).

TABLE 1

| | Maximum | Mean | Voided | | Voiding | Voiding | |
|---|---|---|---|---|---|---|---|
| | flow rate | flow rate | volume | Voiding | hesitancy | curve | Whether |
| | (ml/s) | (ml/s) | (ml) | time (s) | time (s) | pattern | there is risk |
| Example 1 | 43.1 | 18.8 | 417 | 21 | 2 | Bell-shaped | Normal |
| | | | | | | normal | |
| | | | | | | pattern | |
| Example 2 | 12.7 | 2.3 | 84 | 27 | 6 | Interrupted- | Low risk |
| | | | | | | shaped | group |
| | | | | | | pattern | |
| Example 3 | 10.4 | 1.8 | 260 | 65 | 20 | Compressive | High risk |
| | | | | | | pattern | group |
| Example 4 | 5.3 | 5.0 | 346 | 75 | 5 | Plateau- | High risk |
| | | | | | | shaped | group |
| | | | | | | constrictive | |
| | | | | | | pattern | |

In one embodiment, the examinee information may include information of examinee's age, examinee's gender, examination time zone and disease (prostatic hyperplasia, urinary incontinence, nocturia, overactive bladder symptoms etc.), and the prestored reference result value or reference voiding curve pattern to be compared in the terminal 200 may be set differently depending on the aforementioned examinee information. That is, upon comparing the result values derived in the uroflowmetry apparatus 100, a look up table for classifying the examinee into one of the normal group, low risk group and high risk group may be verified according to the aforementioned examinee information. Accordingly, as the examinee information is reflected when performing the uroflowmetry, there is an effect that a more accurate identification is possible.

However, as in Example 2 of Table 1, when the voided volume of the examinee is not more than 150 ml, an accurate measurement is difficult, and thus the terminal 200 may output a message that it is impossible to analyze when the voided volume of the result value is not more than 150 ml.

Meanwhile, the voiding diary is a record of the examinee's voiding time and voided volume, and is one of the first methods for the examiner to objectively observe the subjective symptoms of the examinee suffering from voiding disorder. However, in the past voiding diary method, there was a problem that when the examinee handwrites the voiding record such as the voiding time and voided volume in the voiding diary for about 3 days using writing tools, the examiner would have to go through the hassle of analyzing the handwritten voiding diary one by one in order to diagnose the examinee's symptoms, and derive the voiding diary parameters such as daytime voiding diary parameters, nighttime voiding diary parameters and 24-hour voiding diary parameters.

In order to resolve the aforementioned problem, the terminal 200 of the present disclosure may receive the result values transmitted in the portable uroflowmetry apparatus 100, automatically write and output the voiding diary based on the received result values, thereby quantitatively record the degree of frequent voiding, nocturia and urgent voiding.

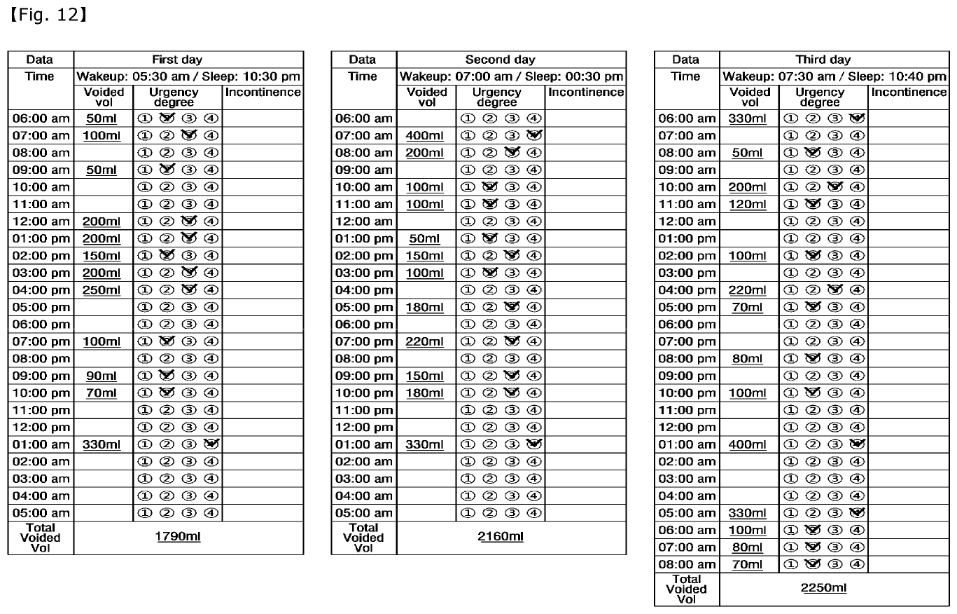

FIG. 12 is a view illustrating the voiding diary being written in the terminal of the portable uroflowmetry apparatus according to an embodiment of the present disclosure, and uroflowmetry and voiding diary writing system and uroflowmetry method using the same.

Referring to FIG. 12, the terminal 200 may derive the number of voids by time zone, voided volume by time zone and urgency degree by time zone based on the result values received from the portable uroflowmetry apparatus 100, and write the voiding diary that includes at least one of the number of voids by time zone, voided volume by time zone and urgency degree by time zone. Here, the number of voids by time zone is the number of voids in each time zone recorded by the examinee, the voided volume by time zone is the voided volume in each time zone recorded by the examinee, and the urgency degree by time zone is a parameter indicating how much urgent the examinee was during voiding, and may be classified into 4 stages, for example (stage 1 being not urgent and stage 4 being very urgent, etc.).

In one embodiment, the number of voids by time zone, voided volume by time zone and urgency degree by time zone may be indicated as a graph for each item, or as a table with numerical figures.

In one embodiment, the terminal 200 of the present disclosure may record the number of voids by time zone, voided volume by time zone and urgency degree by time zone in the voiding diary in a frequency-volume chart (FVC) method.

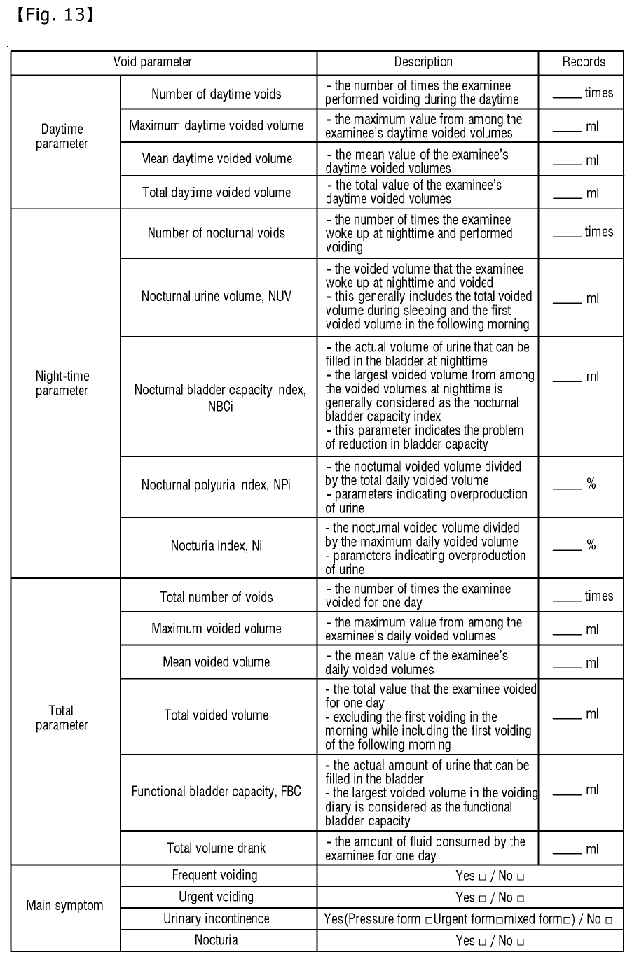

FIG. 13 is a view illustrating the voiding diary that includes voiding diary parameters, being written in the terminal of the portable uroflowmetry apparatus according to an embodiment of the present disclosure, and uroflowmetry and voiding diary writing system and uroflowmetry method using the same.

Referring to FIG. 13, the terminal 200 may write the voiding diary that includes the voiding diary parameters.

That is, the terminal 200 of the present disclosure derives various voiding diary parameters based on the result values received in the portable uroflowmetry apparatus 100, and writes and outputs the voiding diary that includes the voiding diary parameters, and thus the examiner may identify not only the voiding record of the examinee but also the various voiding diary parameters at a glance, and thus is able to make a more accurate and quicker diagnosis regarding symptoms such as the frequent voiding, nocturia, urgent voiding and the like of the examinee.

Here, the voiding diary parameters refers to parameters related to the examinee's voiding, and these voiding diary parameters may include at least one of the daytime voiding diary parameters, nighttime voiding diary parameters and 24-hours voiding diary parameters.

The daytime voiding diary parameters refer to parameters regarding the urinations that the examinee performed between, for example, 6 am to 5 pm, and such daytime voiding diary parameters may include at least one of the number of daytime voids, maximum daytime voided volume, mean daytime voided volume, and total daytime voided volume.

Here, the number of daytime voids refers to the number of times the examinee performed voiding during the daytime, and the maximum daytime voided volume refers to the maximum value from among the examinee's daytime voided volumes, the mean daytime voided volume refers to the mean value of the examinee's daytime voided volumes, and the total daytime voided volume refers to the total value of the examinee's daytime voided volumes.

The nighttime voiding diary parameters refer to the parameters related to the voiding that the examinee performed at nighttime, for example, between 6 pm and 5 am the next day, and these nighttime voiding diary parameters may include at least one of the number of nocturnal voids, nocturnal urine volume (NUV), nocturnal bladder capacity index (NBCi), nocturnal polyuria index (NPi) and nocturia index (Ni).

Here, the number of nocturnal voids refers to the number of times the examinee woke up at nighttime and performed voiding, and the nocturnal urine volume (NUV) refers to the voided volume that the examinee woke up at nighttime and voided, and this generally includes the total voided volume during sleeping and the first voided volume in the following morning. The nocturnal bladder capacity index (NBCi) refers to the actual volume of urine that can be filled in the bladder at nighttime, and the largest voided volume from among the voided volumes at nighttime is generally considered as the nocturnal bladder capacity index (NBCi), and this parameter indicates the problem of reduction in bladder capacity. The nocturnal polyuria index and nocturia index are parameters indicating overproduction of urine. The nocturnal polyuria index refers to the nocturnal voided volume divided by the total daily voided volume, and the nocturia index refers to the nocturnal voided volume divided by the maximum daily voided volume.

The 24-hour voiding diary parameters refer to the parameters related to the voiding that the examinee performed for one day, for example, from 6 am until 5 am the next morning, and such 24-hour voiding diary parameters may include at least one of the total daily number of voids, maximum daily voided volume, mean daily voided volume, total daily voided volume, functional bladder capacity (FBC) and total daily amount of fluid intake.

Here, the total daily number of voids refers to the number of times the examinee voided for one day, and the maximum daily voided volume refers to the maximum value from among the examinee's daily voided volumes, and the mean daily voided volume refers to the mean value of the examinee's daily voided volumes, the total daily voided volume refers to the total value that the examinee voided for one day, but generally, excluding the first voiding in the morning while including the first voiding of the following morning. The functional bladder capacity is different from maximal bladder capacity. The functional bladder capacity refers to the actual amount of urine that can be filled in the bladder, and generally, the largest voided volume in the voiding diary is considered as the functional bladder capacity. The total daily amount of fluid intake refers to the amount of fluid consumed by the examinee for one day. In one embodiment, the examinee may input the total daily amount of fluid intake through the display 124 of the portable uroflowmetry apparatus in a touch input manner, or input directly into the terminal 200.

In one embodiment, the terminal 200 may write the voiding diary that includes determination regarding main symptoms. For example, the voiding diary being written by the terminal 200 may include determinations regarding whether there is frequent voiding, urgent voiding, urinary incontinence (in this case, whether it is in the abdominal pressure form, urgent voiding form, or mixed form), and nocturia, based on the result values received in the portable uroflowmetry apparatus 100.

In one embodiment, the terminal 200 may derive a standard deviation or standard error regarding each voiding diary parameter, so that the examiner can observe subtle changes of the voiding diary parameter, and record them in the voiding diary.

In one embodiment, the terminal 200 may derive the mean result value, that is the mean value of the result values measured for a predetermined period of time, for example, for 3 days, and may derive the voiding diary parameters (daytime voiding diary parameter, nighttime voiding diary parameter, or 24-hour voiding diary parameter) based on the derived mean result value.

In one embodiment, the voiding diary being written and output in the terminal 200 may include at least one of the examination date and examination time zone, and the result values such as the maximum flow rate of the fluid, mean flow rate of the fluid, voided volume, voiding time and voiding hesitancy time.

In one embodiment, the terminal 200 may determine whether the examinee has a disease such as frequent voiding, nocturia and interrupted voiding, upon comparing the result value received in the portable uroflowmetry apparatus 100 with the prestored reference result value, or based on the derived voiding diary parameters. Here, frequent voiding refers to a state where the number of voids increases abnormally, and nocturia refers to a state where the number of voids increases abnormally at nighttime, and interrupted voiding refers to the flow of urine being interrupted during voiding.

Here, the prestored reference result values refer to the maximum flow rate, mean flow rate, voided volume, voiding time and hesitancy time observed in a normal case, and the maximum flow rate, mean flow rate, voided volume, voiding time and hesitancy time observed in an abnormal case (preferably per each disease).

In another embodiment, the terminal 200 may derive a graph regarding the examinee's voiding curve pattern using the received result values, and the voiding diary being written and output in the terminal 200 may include such a voiding curve pattern.

Further, the terminal 200 may compare the derived voiding curve pattern of the examinee with the prestored reference voiding curve pattern, and determine as one of the normal group, low risk group, and high risk group, and then include it in the voiding diary and output the same.

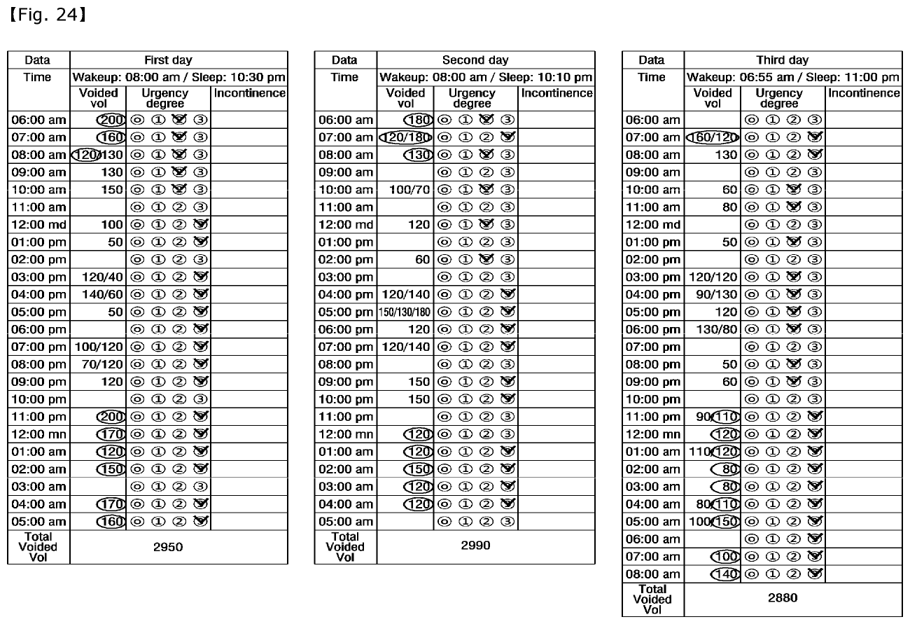

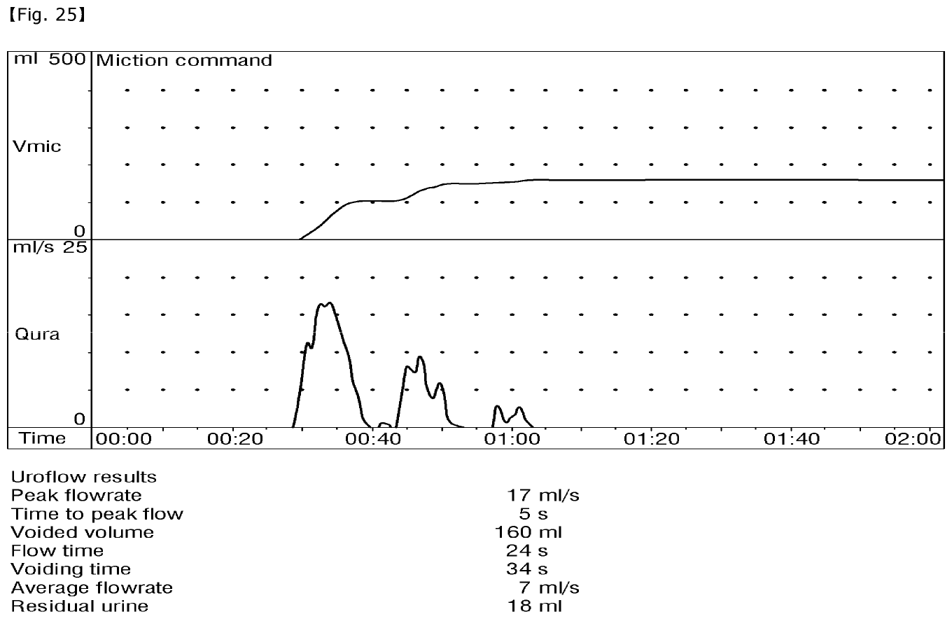

FIG. 24 is a view illustrating the voiding diary actually written in the terminal of the portable uroflowmetry apparatus according to an embodiment of the present disclosure, and uroflowmetry and voiding diary writing system and uroflowmetry method using the same, and FIG. 25 is a view illustrating the actual voiding curve pattern included in the voiding diary being written in the terminal of a portable uroflowmetry apparatus according to an embodiment of the present disclosure, and uroflowmetry and voiding diary writing system and uroflowmetry method using the same.

FIGS. 24 and 25 illustrate a voiding diary that includes a voiding curve pattern, that has been written using a voiding diary writing system of the present disclosure, of a 75-year old female examinee who visited the hospital due to bladder pain and severe frequent voiding. The most uncomfortable symptom that the examinee had was the pain in the vagina and humerus when she felt the urge to urinate, and after voiding, the symptom would get better. When she felt the urge to urinate, she could not bear the pain, so she went to the toilet frequently, that is, at least 10 times of nocturia. The examinee also said she had urgent voiding, urgent urinary incontinence and abdominal straining urinary incontinence as well.

Referring to FIG. 24, according to the voiding diary written in the terminal, at least 24 times of daily voiding including 8.6 times of nocturia were derived, with the mean voided volume per voiding being 114 ml, and 24 hour mean urine volume being 2,746 ml. The voided volume and voiding interval pattern at daytime were equivalent to nighttime. Referring to FIG. 25, according to the voiding curve pattern derived in the terminal, urine flow showed an intermittent pattern, and residual volume was small. According to the record, 24-hour voided volume was 2,746 ml, which means polyuria is observed, and accordingly, the examiner may apply a behavioral therapy for the examinee to restrict the nighttime fluid intake so as to reduce the daily voided volume to about 1,500 ml˜1,700 ml.

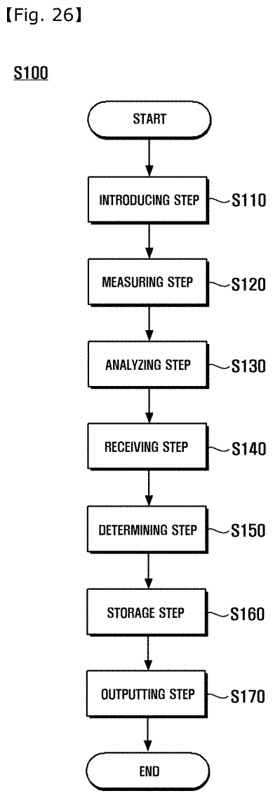

Referring to FIG. 26, the configuration and operations of a uroflowmetry method (S100) according to an embodiment of the present disclosure will be described hereinafter.

FIG. 26 is a flowchart of a uroflowmetry method according to an embodiment of the present disclosure.

Referring to FIG. 26, the uroflowmetry method according to an embodiment of the present disclosure may include introducing step (S110), measuring step (S120), analyzing step (S130), receiving step (S140), determining step (S150), storing step (S160), and outputting step (S170).

The introducing step (S110) is a step of introducing the fluid into the portable uroflowmetry apparatus 100.

Fluid in the present disclosure refers to the examinee's urine being introduced into the tube part 110 of the portable uroflowmetry apparatus 100 by the examinee's voiding activity, and as the present disclosure measures the flow rate of the examinee's urine introduced over time, the result value for diagnosing the lower urinary tract dysfunction is derived.

The measuring step (S120) is a step of measuring the flow rate of the fluid introduced over time into the portable uroflowmetry apparatus 100.

In more detail, the measuring step (S120) is a step of analyzing the degree of rotation motion of the rotation part 122 to measure the flow rate of the fluid introduced over time. The flow rate of the fluid introduced over time is measured through the tachometer that is connected to the rotation shaft of the rotation part 122 to make a pulse signal for the rotation motion of the rotation shaft, or the rotary encoder connected to the rotation shaft to digitally signal the rotation motion of the rotation shaft.

The analyzing step (S130) is a step of analyzing the measured flow rate of the fluid and deriving the result value.

Here, the result values derived at the analyzing step (S130) include the maximum flow rate (Qmax), mean flow rate, voided volume, voiding time and hesitancy time, and here, the maximum flow rate refers to the maximum value of the fluid flow rate, the mean flow rate refers to the mean value of the voided fluid flow rate, the voided volume refers to the total amount of the voided fluid, the voiding time refers to the total period of time the examinee spent in voiding, and voiding hesitancy time refers to the period of time delayed until the examinee started voiding.

In one embodiment, the analyzing step (S130) may derive the mean result value, that is the mean value of the result values measured for the predetermined period of time, for example, for 3 days. Generally, the examiner lets the examinee record the parameters such as the voided volume and the like in the voiding diary for 3 days, and then determines the examinee's symptoms based on the record, but in this method, there is a hassle that the examinee has to record every urination, and the examiner has the inconvenience of separately calculating the 3 days' result values. Accordingly, the analyzing step (S130) analyzes the flow rate over time of the fluid measured for the predetermined period of time, to derive the result values of the maximum flow rate, mean flow rate, voided volume, voiding time and voiding hesitancy time, respectively, and then calculates the mean value of those results values, to derive the mean result value.

The receiving step (S140) is a step for the terminal 200 to receive the result value derived and transmitted from the portable uroflowmetry apparatus 100.

The determining step (S150) is a step of comparing the derived result value with the prestored reference result value, and determining as one of the normal group, low risk group, and high risk group.

The storing step (S160) is a step of storing the result value derived and received at the analyzing step (S130), and storing the result of determination at the determining step (S150).

The outputting step (S170) is a step of writing and outputting the voiding information based on the examination date, examination time zone and derived result values, and of outputting the result of determination at the determining step (S150).

In one embodiment, the voiding information being written and output at the outputting step (S170) may include at least one of the examination date and examination time zone, and the result values such as the maximum flow rate of the fluid, mean flow rate of the fluid, voided volume, voiding time and voiding hesitancy time.

However, since when the voided volume of the examinee is not more than 150 ml, it is difficult to perform an accurate measurement, when a voided volume, of the result values, is not more than 150 ml, the outputting step (S170) may output the message saying that analyzing is impossible.

In another embodiment, the determining step (S150) may derive a graph regarding the voiding curve pattern of the examinee using the received result value, and may compare the derived voiding curve pattern of the examinee with the prestored reference voiding curve pattern, and determine as one of the normal group, low risk group and high risk group.

Here, the voiding information being written and output at the outputting step (S170) may include the voiding curve pattern, and the outputting step (S170) may compare the derived voiding curve pattern and the reference voiding curve pattern, and output the determined result.

Referring to FIG. 14, the determining step (S150) may derive the graph regarding the examinee's voiding curve pattern for the predetermined period of time, for example, 3 days, using the result values measured for 3 days, and average the same to derive the mean voiding curve pattern. Thereafter, the determining step (S150) may compare the derived mean voiding curve pattern with the prestored reference voiding curve pattern, and determine as one of the normal group, low risk group and high risk group, and the examiner may use the derived mean voiding curve pattern in diagnosing the symptoms.

Here, the prestored reference voiding curve patterns may include the bell-shaped normal pattern, tower-shaped superflow pattern, compressive pattern, plateau-shaped constrictive pattern, interrupted-shaped pattern and abdominal straining pattern, and since these were already descried hereinabove, repeated description will be omitted.

In another embodiment, the determining step (S150) may determine the examinee as one of the normal group, low risk group and high risk group using the prelearned artificial neural network.

In more detail, the artificial neural network may sustainably learn the reference result value and reference voiding curve pattern (preferably, the result value and voiding curve pattern derived from actual disease cases), and upon receiving the result value of the examinee, the determining step (S150) may determine the examinee as one of the normal group, low risk group and high risk group using the prelearned artificial neural network. Meanwhile, here, the artificial neural network may be provided as the deep convolutional neural network (DCNN), but there is no limitation thereto.

The embodiments disclosed in the present specification belong to the same technology field, and the components constituting one embodiment may be combined with the components constituting other embodiments to constitute a new embodiment.

The scope of protection of the present disclosure is not limited to the disclosure of embodiments and expressions described hereinabove. Further, it is added once again that the scope of protection of the present disclosure cannot be limited due to obvious changes or substitutions in the technology field to which the present disclosure pertains.

In describing the embodiments of the present disclosure, each of the “parts”, “modules” or “steps” can be implemented through a processor or memory. The processor should be construed broadly to include general purpose processors, central processing units (CPUs), microprocessors, digital signal processors (DSPs), controllers, microcontrollers, state machines, and the like. In some environments, a processor may refer to an application specific semiconductor (ASIC), a programmable logic device (PLD), a field programmable gate array (FPGA), and the like. A processor may refer to a combination of processing devices, such as, for example, a combination of a DSP and a microprocessor, a combination of a plurality of microprocessors, a combination of one or more microprocessors in combination with a DSP core, or any other such configurations.

The memory of the present disclosure should be construed broadly to include any electronic component capable of storing electronic information. The memory may refer to various kinds of processor-readable media such as random access memory (RAM), read-only memory (ROM), non-volatile random access memory (NVRAM), programmable read-only memory (PROM), erase-programmable read only memory (EPROM), electrically erasable PROM (EEPROM), flash memory, magnetic or optical data storage, and registers, etc. If the processor can read information from the memory or can record information in the memory, the memory is said to be in an electronic communication state with the processor, and each of the “part”, “module”, or “step” may be implemented through a program or application that is based on the processor and memory in an electronic communication state.

What is claimed is:

1. A portable uroflowmetry apparatus comprising:
a tube part where fluid flows; and
an examination module that is connected to the tube part, and that measures a flow rate of the introduced fluid over time and analyzes the measured flow rate of the fluid, to derive a result value,
wherein the examination module comprises an inlet that is connected to one side of the tube part and to which the fluid is introduced, a rotation part that is rotated by the introduced fluid, a controller that measures the flow rate of the introduced fluid over time by analyzing a rotation degree of the rotation part, and analyzes the measured flow rate of the fluid, to derive the result value, and an outlet that is connected to the other side of the tube part and through which the measured fluid is discharged.

2. The portable uroflowmetry apparatus according to claim 1,
wherein the controller comprises a measurement part that measures the flow rate of the introduced fluid over time, an analyzing part that analyzes the measured flow rate of the fluid, to derive the result value, and a determining part that compares the derived result value with a prestored reference result value, to determine as one of a normal group, low risk group, and high risk group.

3. The portable uroflowmetry apparatus according to claim 1,
wherein the result value comprises at least one of a maximum flow rate, mean flow rate, voided volume, voiding time and voiding hesitancy time of the fluid, and
the examination module further comprises a display that displays at least one of an examination date, examination time zone and the result value.

4. The portable uroflowmetry apparatus according to claim 3,
wherein the controller derives a mean result value, that is a mean value of the result value measured for a predetermined period of time, and
the display displays the mean result value.

5. The portable uroflowmetry apparatus according to claim 1,
wherein the examination module further comprises a button for setting an examination time zone so as to differentiate between daytime zone and nighttime zone.

6. The portable uroflowmetry apparatus according to claim 1,
wherein the tube part is formed in a shape that can be reduced in volume or length.

7. A uroflowmetry system using a portable uroflowmetry apparatus, the system comprising:
a portable uroflowmetry apparatus that measures a flow rate of introduced fluid over time, analyzes the measured flow rate of the fluid, to derive a result value, and transmits the derived result value in a wired or wireless manner; and
a terminal that receives the result value transmitted from the portable uroflowmetry apparatus, derives a voiding curve pattern based on the received result value, and writes and outputs voiding information based on at least one of the result value and the voiding curve pattern.

8. The uroflowmetry system using a portable uroflowmetry apparatus, according to claim 7,
wherein the result value comprises at least one of a maximum flow rate, mean flow rate, voided volume, voiding time and voiding hesitancy time of the fluid, and
the voiding information comprises at least one of an examination date, examination time zone, the result value and the voiding curve pattern.

9. The uroflowmetry system using a portable uroflowmetry apparatus, according to claim 8,
wherein the terminal compares the received result value or the voiding curve pattern with a prestored reference result value or a reference voiding curve pattern, and then determines and outputs as one of a normal group, low risk group and high risk group.

10. The uroflowmetry system using a portable uroflowmetry apparatus, according to claim 8,
wherein the reference voiding curve pattern comprises a bell-shaped normal pattern, tower-shaped superflow pattern, compressive pattern, plateau-shaped constrictive pattern, interrupted-shaped pattern and abdominal straining pattern.

11. The uroflowmetry system using a portable uroflowmetry apparatus, according to claim 8,
wherein the terminal derives a mean result value, that is a mean value of the result value measured for a predetermined period of time, compares the mean result value with a prestored reference result value, and then determines and outputs as one of a normal group, low risk group, and high risk group.

12. The uroflowmetry system using a portable uroflowmetry apparatus, according to claim 8,
wherein the terminal derives a mean voiding curve pattern, that is a mean value of the voiding curve pattern measured for a predetermined period of time, and compares the mean voiding curve pattern with a prestored reference voiding curve pattern, and then determines and outputs as one of a normal group, low risk group, and high risk group.

13. A voiding diary writing system using a portable uroflowmetry apparatus, the system comprising:
a portable uroflowmetry apparatus that measures a flow rate of introduced fluid over time, analyzes the measured flow rate of the fluid, to derive a result value, and transmits the derived result value in a wired or wireless manner; and
a terminal that receives the result value transmitted from the portable uroflowmetry apparatus, and writes and outputs a voiding diary based on the received result value.

14. The voiding diary writing system using a portable uroflowmetry apparatus, according to claim 13,
wherein the terminal writes the voiding diary that comprises at least one of the number of voids by time zone, voided volume by time zone, and urgency degree by time zone.

15. The voiding diary writing system using a portable uroflowmetry apparatus, according to claim 13,
wherein the terminal writes the voiding diary that comprises a voiding diary parameter, and
the voiding diary parameter comprises at least one of a daytime voiding diary parameter, nighttime voiding diary parameter and 24-hour voiding diary parameter.

16. The voiding diary writing system using a portable uroflowmetry apparatus, according to claim 15,
wherein the daytime voiding diary parameter comprises at least one of the number of daytime voids, maximum daytime voided volume, mean daytime voided volume and total daytime voided volume.

17. The voiding diary writing system using a portable uroflowmetry apparatus, according to claim 15,
wherein the nighttime voiding diary parameter comprises at least one of the number of nocturnal voids, nocturnal voided volume, nocturnal bladder capacity, nocturnal polyuria parameter, and nocturia parameter.

18. The voiding diary writing system using a portable uroflowmetry apparatus, according to claim 15,
wherein the 24-hour voiding diary parameter comprises at least one of the number of daily voids, maximum daily voided volume, mean daily voided volume, total daily voided volume, and functional bladder capacity.

19. The voiding diary writing system using a portable uroflowmetry apparatus, according to claim 15,
wherein the terminal derives a mean result value, that is a mean value of the result value measured for a predetermined period of time, and derives the voiding diary parameter based on the mean result value.

20. The voiding diary writing system using a portable uroflowmetry apparatus, according to claim 13,
wherein the portable uroflowmetry apparatus comprises a tube part where fluid flows, and an examination module that is connected to the tube part, and that measures a flow rate of the introduced fluid over time and analyzes the measured flow rate of the fluid, to derive a result value, and transmits the derived result value in a wired or wireless manner, and
the examination module comprises a first button that can set an examination time zone so as to differentiate between daytime voiding and nocturnal voiding.
